(19) United States
(12) Patent Application Publication
(10) Pub. No.: US 2015/0099287 A1
(43) Pub. Date: Apr. 9, 2015

(54) BIOGAS GENERATION SYSTEM AND METHOD FOR GENERATING BIOGAS AND CARBON DIOXIDE REDUCTION PRODUCT USING THE SAME

(71) Applicant: Panasonic Corporation, Osaka (JP)

(72) Inventors: SATOSHI YOTSUHASHI, Osaka (JP); MASAHIRO DEGUCHI, Osaka (JP); HIROSHI HASHIBA, Osaka (JP); TAKEYUKI SEKIMOTO, Osaka (JP); YUKA YAMADA, Nara (JP)

(21) Appl. No.: 14/482,165

(22) Filed: Sep. 10, 2014

(30) Foreign Application Priority Data
Oct. 3, 2013 (JP) 2013-208009

(51) Int. Cl. C12P 5/02

(52) U.S. Cl. CPC C12P 5/023
USPC 435/167; 435/294.1

(57) ABSTRACT

Provided is a biogas generation system comprising a fermenter for generating a biogas containing carbon dioxide and methane by decomposing an organic waste by the action of methane bacteria; a biogas refinery for condensing the methane contained in the biogas by dissolving the carbon dioxide contained in the generated biogas in a liquid; and a photoelectrochemical device for generating methane, carbon monoixide, or formic acid from the carbon dioxide dissolved in the liquid. The photoelectrochemical device comprises a cathode chamber for storing a first electrolyte solution containing the carbon dioxide dissolved in the liquid; an anode chamber for storing a second electrolyte solution; a solid electrolyte membrane; a cathode electrode provided in the cathode chamber; an anode electrode provided in the anode chamber; and an external power supply for applying a negative voltage and a positive voltage to the cathode electrode and the anode electrode, respectively.

### BACKGROUND

1. Field of the Invention

The present invention relates to a biogas generation system for effectively using carbon dioxide accompanying methane generation by the action of methane bacteria. The present invention also relates a method for generating a biogas and a carbon dioxide reduction product using the biogas generation system.

2. Description of the Related Art

Recently, methane fermentation of generating methane usable as a biogas has been attracting attention. Japanese Patent Laid-Open Publication No. 2002-239508A discloses a basic biogas generation system.

The biogas contains carbon dioxide and hydrogen sulfide as impurities. Hydrogen sulfide is derived from sulfur ingredients contained in fermentation raw materials. Japanese Patent Laid-Open Publication No. 2008-255209A discloses a technique to raise the methane concentration of the biogas. In particular, a biogas containing impurities is dispersed in the form of microbubbles or nanobubbles in an absorbing liquid such as water or discharged water to dissolve carbon dioxide and hydrogen sulfide in the absorbing liquid. In this way, the impurities such as carbon dioxide and hydrogen sulfide are separated from the biogas to give a purified biogas containing methane in high purity.

Japanese Patent Laid-Open Publication No. 2012-090623A discloses a renewable energy multi-stage usage system.

WO 2011/067873A discloses a method for reducing carbon dioxide, and a carbon dioxide reduction catalyst and a carbon dioxide reduction apparatus used therein.

### SUMMARY

The present invention provides a biogas generation system comprising:
a fermenter for generating a biogas containing carbon dioxide and methane by decomposing an organic waste by the action of methane bacteria;
a biogas refinery for condensing the methane contained in the biogas by dissolving the carbon dioxide contained in the generated biogas in a liquid; and
a photoelectrochemical device for generating at least one of carbon dioxide reduction products selected from the group consisting of methane, carbon monoixide, and formic acid from the carbon dioxide dissolved in the liquid, wherein
the photoelectrochemical device comprises:
   a cathode chamber for storing a first electrolyte solution containing the carbon dioxide dissolved in the liquid;
   an anode chamber for storing a second electrolyte solution;
   a solid electrolyte membrane interposed between the cathode chamber and the anode chamber;
   a cathode electrode provided in the cathode chamber so as to be in contact with the first electrolyte solution;
   an anode electrode provided in the anode chamber so as to be in contact with the second electrolyte solution; and
   an external power supply for applying a negative voltage and a positive voltage to the cathode electrode and the anode electrode, respectively.

The present invention provides a biogas generation system and method for generating a biogas for effectively using carbon dioxide dissolved in a liquid as impurities.

### BRIEF DESCRIPTION OF THE DRAWINGS

FIG. 1 shows a schematic view of a biogas generation system according to an embodiment; and

FIG. 2 shows a schematic view of a photoelectrochemical device according to the embodiment.

### DETAILED DESCRIPTION OF THE EMBODIMENTS

The embodiments of the present invention will be described in detail with reference to the drawings.

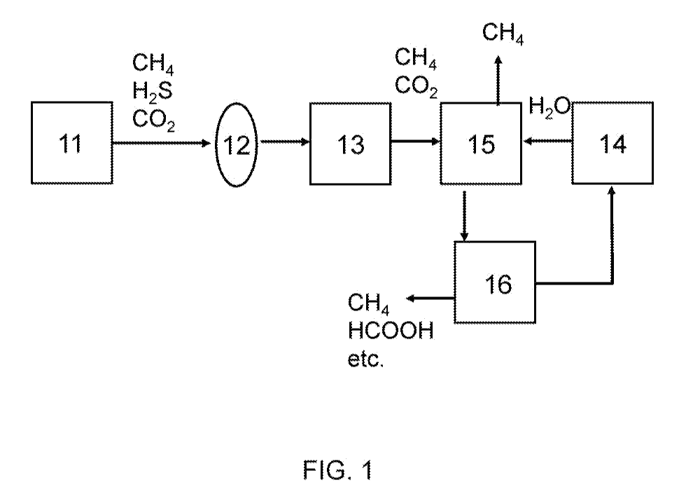

FIG. 1 shows a schematic view of a biogas generation system 10 according to an embodiment.

The biogas generation system 10 comprises a fermenter 11, a biogas refinery 15, and a photoelectrochemical device 16. The biogas generation system 10 may further comprise a gas bag 12, a desulfurization device 13, and a water tank 14.

The fermenter 11 is used to obtain methane by fermenting an organic waste. An example of the organic waste is garbage, livestock excreta, sewerage sludge, or septic tank sludge. In the fermenter 11 in which the organic waste has been put, the organic waste is fermented in an anaerobic condition by the action of the methane bacteria to generate a biogas. The generated biogas contains methane, carbon dioxide, and hydrogen sulfide. Carbon dioxide and hydrogen sulfide are impurities. The generated biogas is stored in the gas bag 12.

The gas bag 12 serves as a buffer in the gas exhaust. If the gas bag 12 has a volume equivalent to approximately one day's gas exhaust amount, the gas bag 12 serves as a buffer. The gas bag 12 is made of metal or plastic. The biogas stored in the gas bag 12 is supplied to the desulfurization device 13.

The desulfurization device 13 removes hydrogen sulfide contained in the biogas. Hydrogen sulfide has to be removed, since the hydrogen sulfide has harmful effects on the photoelectrochemical device 16. An example of the desulfurization method is a hydrodesulfurization method. Hydrogen sulfide is required to be removed, before the biogas is supplied to the photoelectrochemical device 16. This is because hydrogen sulfide contained in the biogas may corrode an anode electrode 211 and or a cathode electrode 221 included in the photoelectrochemical device 16.

The biogas refinery 15 removes carbon dioxide contained in the biogas. In particular, in the biogas refinery 15, the biogas containing carbon dioxide is brought into contact with a high-pressure liquid to dissolve carbon dioxide in the high-pressure liquid. In this way, carbon dioxide contained in the biogas is removed. As a result, the biogas refinery 15 purifies the biogas to give a methane gas.

Generally, the biogas is purified in the biogas refinery 15 under a condition where the liquid contained in the biogas refinery 15 has a low temperature and where the inside of the biogas refinery 15 has a high pressure. In a method for purifying the biogas, for example, the biogas is introduced from a lower part of the biogas refinery 15, and the liquid is drizzled from an upper part of the biogas refinery 15 to the biogas. In a different method, the biogas in the form of fine bubbles such as microbubbles or nanobubbles may be introduced into the liquid.

In the biogas refinery 15, the biogas from which hydrogen sulfide has been removed in the desulfurization device 13 may be compressed in the biogas refinery 15. In a different example, a device for compressing the biogas may be provided between the desulfurization device 13 and the biogas refinery 15. It is desirable that the inside of the biogas refinery 15 has a pressure of 0.50×10⁶ Pa or more. More desirably, the pressure is 1.013×10⁶ Pa or more.

The liquid may be supplied from the water tank 14 to the biogas refinery 15. It is desirable that the liquid has a temperature of not more than 15 degrees Celsius. More desirably, the temperature is not more than 10 degrees Celsius.

The liquid to be supplied to the biogas refinery 15 is stored in the water tank 14. The materials of the water tank 14 are not limited, as long as water is stored in the water tank 14. It is desirable that the water tank has about ten times or more the volume of the biogas exhausted in an hour.

The water tank 14 comprises an inlet through which the liquid is supplied to the biogas refinery 15 and an outlet through which the liquid is supplied from the photoelectrochemical device 16. It is desirable that the liquid stored in the water tank 14 is an electrolyte solution, since the liquid stored in the water tank 14 is used for the photoelectrochemical device 16, which will be described later. An example of the liquid stored in the water tank 14 is a potassium chloride aqueous solution, a sodium chloride aqueous solution, a potassium hydrogen carbonate aqueous solution, or a sodium hydrogen carbonate aqueous solution. Another example may be seawater which occurs in nature and condensed seawater.

(Photoelectrochemical Device 16)

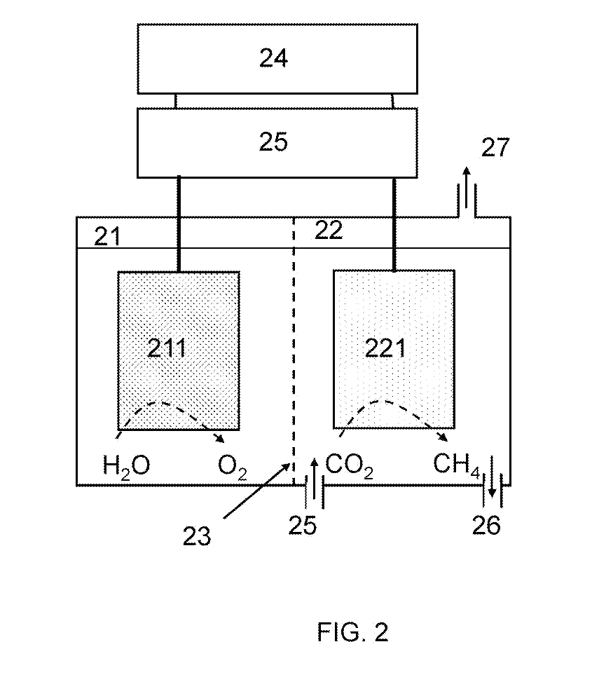

FIG. 2 shows a schematic view of the photoelectrochemical device 16 for reducing carbon dioxide.

The photoelectrochemical device 16 comprises an anode chamber 21, a cathode chamber 22, a solid electrolyte membrane 23, and an external power supply 29.

The anode chamber 21 holds a first electrolyte solution. The first electrolyte solution held in the anode chamber 21 may be the same as a second electrolyte solution held in the cathode chamber 22. Desirably, the first electrolyte solution is different from the second electrolyte solution. An anode electrode 211 is provided in the anode chamber 21 so as to be in contact with the first electrolyte solution. A voltage may be applied to the anode electrode 211 from the external power supply 29 to generate oxygen on the surface of the anode electrode 211. The materials of the anode electrode 211 are not limited, as far as an oxidation reaction of water occurs stably. An example of the materials of the anode electrode is platinum, gold, or glassy carbon.

The cathode chamber 22 holds the second electrolyte solution. The second electrolyte solution held in the cathode chamber 22 contains the liquid supplied from the biogas refinery 15. The cathode chamber 22 comprises an inlet 25 through which the liquid is supplied from the biogas refinery 15 to the inside of the cathode chamber 22, a drain outlet 26 through which the liquid contained in the cathode chamber 22 is drained to the water tank 14, and an exhaust outlet 27. The liquid circulates among the biogas refinery 15, the cathode chamber 22, and the water tank 14. The liquid may always circulate. Instead, the liquid may circulate at a predetermined interval. The predetermined period may depend on the volume of the liquid held in the cathode chamber 22 and on the surface area of the anode electrode.

A cathode electrode 221 is disposed in the cathode chamber 22 so as to be in contact with the electrolyte solution. The reduction product of carbon dioxide contained in the electrolyte solution depends on the materials of the cathode electrode 221. When the cathode electrode 221 is made of copper or an alloy thereof, methane may be generated as the reduction product of carbon dioxide. When the cathode electrode 221 is made of gold or silver, carbon monoxide may be generated as the reduction product of carbon dioxide. When the cathode electrode 221 is made of indium or tin, formic acid may be generated as the reduction product of carbon dioxide. As just described, the reduction product of carbon dioxide is an organic substance or carbon monoxide.

It is desirable that the anode chamber 21 and the cathode chamber 22 are separated from each other so that oxygen generated in the anode chamber 21 does not mix with the carbon dioxide reduction product generated in the cathode chamber 22. Since ions are required to move between the anode chamber 21 and the cathode chamber 22, it is desirable that the anode chamber 21 and the cathode chamber 22 are separated from each other using the solid electrolyte membrane 23 which is interposed therebetween.

It is desirable that the anode chamber 21 and the cathode chamber 22 are each provided with a stirring mechanism of the electrolyte solution to promote an oxidation-reduction reaction. An example of the stirring mechanism is a magnetic stirrer.

The external power supply 29 applies a negative voltage and a positive voltage to the cathode electrode 221 and the anode electrode 211, respectively. An example of the external power supply 29 is a potentiostat, a solar battery 24, or a storage battery.

The output electric power output from the solar battery 24 may be varied depending on the weather. For this reason, when the solar battery 24 is used as the external power supply 29, a rectifier circuit 28 may be provided to maintain an electric current constant. It is effective to use the storage battery together with the solar battery 24 when sufficient sunlight is not obtained.

(Method for Reducing Carbon Dioxide)

Carbon dioxide dissolved in the electrolyte solution is reduced on the cathode electrode 221. An energy necessary for the reduction is supplied from the external power supply 29. Carbon dioxide is reduced on the cathode electrode 221, whereas water is oxidized on the anode electrode 211. The oxidation-reduction reaction occurs so as to balance stoichiometrically.

A gaseous component of the carbon dioxide reduction product generated on the cathode electrode 221 is extracted through the exhaust outlet 27 provided in the cathode chamber 22. On the other hand, a liquid component of the carbon dioxide reduction product generated on the cathode electrode 221 is generated in the form of being dissolved in the liquid. For this reason, the liquid component is extracted through a condensation step afterwards. An example of the gaseous component is methane. An example of the liquid component is an acid or alcohol. An example of the condensation step is a condensation process of formic acid used generally in chemical plants.

### EXAMPLE

Hereinafter, the present invention will be described in more detail with reference to the following example.

A container having a volume of 200 liters was prepared as the fermenter 11. Garbage was prepared as an organic waste. Garbage (3 kilograms) having a solid concentration of 20% was thrown to the fermenter 11 together with methane bacteria daily for 20 days. The temperature in the fermenter 11 was maintained at 40 degrees Celsius. The garbage was scrambled up together with methane bacteria until methane is generated due to stable fermentation. In this way, a biogas was generated in the fermenter 11.

Then, the biogas generated in the fermenter 11 was supplied to the gas bag 12 (capacity: 400 liters). The following Table 1 shows the analysis result of the biogas in the gas bag 12.

TABLE 1

| Gas | Volume ratio |
|---|---|
| Methane | 58% |
| Carbon dioxide | 38% |
| Hydrogen sulfide | 0.15% (1,500 ppm) |
| Residual gas such as nitrogen | 3.85% |

Then, a flow meter was provided between the gas bag 12 and the desulfurization device 13 to control the flow rate of the biogas which was supplied from the gas bag 12 to the desulfurization device 13. The flow rate of the biogas was 20 liters an hour.

Hydrogen sulfide contained in the biogas supplied from the gas bag 12 was removed in the desulfurization device 13.

The biogas from which hydrogen sulfide had been removed was supplied from the desulfurization device 13 to the biogas refinery 15. The biogas supplied to the biogas refinery 15 was compressed in the biogas refinery 15. On the other hand, carbon dioxide contained in the biogas supplied to the biogas refinery 15 was dissolved in the liquid supplied from the water tank 14 at 0.911925 MPa under a temperature of 5 degrees Celsius. The liquid supplied from the water tank 14 was a potassium chloride aqueous solution having a concentration of 0.5 M. Methane contained in the biogas was extracted through an outlet provided in the upper of the biogas refinery 15. Methane extracted through the outlet had a purity of 95%.

The liquid in which carbon dioxide contained in the biogas was dissolved was supplied from the biogas refinery 15 to the photoelectrochemical device 16.

Hereinafter, the photoelectrochemical device 16 is described. The photoelectrochemical device 16 shown in FIG. 2 was fabricated. The elements included in the photoelectrochemical device 16 will be described below.

Cathode electrode: Copper sheet of 1 m² (three sheets were used)

Anode electrode: Carbon sheet of 1 m² (three sheets were used)

First electrolyte solution: 0.5 M potassium chloride aqueous solution

Second electrolyte solution: 0.5 M potassium chloride aqueous solution

Solid electrolyte membrane: Nafion film (available from DuPont)

External power supply: Solar battery (electric generation area: 18 m², product of Panasonic Corporation).

The anode chamber 21 and the cathode chamber 22 each had a capacity of 100 liters. The anode chamber 21 and the cathode chamber 22 were each provided with a screw as a stirring mechanism. The cathode chamber 22 was provided with the inlet 25 through which the potassium chloride aqueous solution in which carbon dioxide was dissolved was supplied from the biogas refinery 15, a drain outlet 26 through which the potassium chloride aqueous solution contained in the cathode chamber 22 was drained to the water tank 14, and an exhaust outlet 27 through which the gaseous component generated in the cathode chamber 22 is discharged to the outside of the photoelectrochemical device 16.

The solar battery was irradiated with sunlight sufficiently. The external power supply 29 was provided with the rectifier circuit 28 to adjust the electric current flowing from the cathode electrode 221 to the anode electrode 211 to 600 amperes.

The photoelectrochemical device 16 was configured to supply the potassium chloride aqueous solution in which carbon dioxide was dissolved was supplied from the biogas refinery 15 through the inlet 25 to the cathode chamber 22. The photoelectrochemical device 16 was configured to supply the potassium chloride aqueous solution contained in the cathode chamber 22 from the cathode chamber 22 through the drain outlet 26 to the water tank 14. The photoelectrochemical device 16 was configured to circulate the potassium chloride aqueous solution using a pump (100 liters/min).

The potassium chloride aqueous solution in which carbon dioxide was dissolved was supplied from the biogas refinery 15 to the cathode chamber 22. An electric potential of 4 volts was applied between the anode electrode 211 and the cathode electrode 221 using the external power supply 29.

The gaseous component generated in the cathode chamber 22 was extracted through the exhaust outlet 27. The liquid component generated in the cathode chamber 22 was extracted through the drain outlet 26. The gaseous component generated in the cathode chamber 22 was extracted through the exhaust outlet 27 at a flow rate of 10.8 liters an hour using a flow meter. The following Table 2 shows the analysis result of the gaseous component.

TABLE 2

| | |
|---|---|
| Methane | 41% |
| Hydrogen | 39% |
| Other gas such as ethylene | 20% |
| or carbon monoxide | |

The biogas generation system 10 was operated under clear sky in July for three hours. As a result, the methane production of the biogas refinery 15 was 36 liters. On the other hand, the methane production of the photoelectrochemical device 16 was 11.7 liters. The hydrogen production of the photoelectrochemical device 16 was 11.4 liters.

The above example reveals that the production amount of a flammable gas such as methane was increased from 36 liters to 59.1 liters, which is equal to about 1.6 times the value of 36 liters.

### INDUSTRIAL APPLICABILITY

The present invention provides a biogas generation system and method for generating a biogas for effectively using carbon dioxide dissolved in a liquid as impurities by using a light energy.

### REFERENTIAL SIGNS LIST

10 biogas generation system
11 fermenter
12 gas bag
13 desulfurization device
14 water tank
15 biogas refinery
16 photoelectrochemical device
21 anode chamber
22 cathode chamber
23 solid electrolyte membrane
24 solar battery
25 inlet
26 drain outlet
27 exhaust outlet
28 rectifier circuit
29 external power supply
211 anode electrode
221 cathode electrode

1. A biogas generation system comprising:
a fermenter for generating a biogas containing carbon dioxide and methane by decomposing an organic waste by the action of methane bacteria;
a biogas refinery for condensing the methane contained in the biogas by dissolving the carbon dioxide contained in the generated biogas in a liquid; and
a photoelectrochemical device for generating at least one of carbon dioxide reduction products selected from the group consisting of methane, carbon monoixide, and formic acid from the carbon dioxide dissolved in the liquid, wherein
the photoelectrochemical device comprises:
a cathode chamber for storing a first electrolyte solution containing the carbon dioxide dissolved in the liquid;
an anode chamber for storing a second electrolyte solution;
a solid electrolyte membrane interposed between the cathode chamber and the anode chamber;
a cathode electrode provided in the cathode chamber so as to be in contact with the first electrolyte solution;
an anode electrode provided in the anode chamber so as to be in contact with the second electrolyte solution; and
an external power supply for applying a negative voltage and a positive voltage to the cathode electrode and the anode electrode, respectively.

2. The biogas generation system according to claim 1, wherein
the cathode electrode contains copper, gold, silver, indium, tin, or alloy thereof.

3. The biogas generation system according to claim 1, wherein
the anode electrode contains platinum, gold, or glassy carbon.

4. The biogas generation system according to claim 1, wherein
the external power supply is a solar battery for converting a light energy into an electric energy.

5. The biogas generation system according to claim 1, wherein
the cathode chamber has a stirring mechanism for stirring the first electrolyte solution.

6. A method for generating a biogas and a carbon dioxide reduction product, the method comprising:
(a) preparing a biogas generation system comprising:
a fermenter for generating the biogas containing carbon dioxide and methane by decomposing an organic waste by the action of methane bacteria;
a biogas refinery for condensing the methane contained in the biogas by dissolving the carbon dioxide contained in the generated biogas in a liquid; and
a photoelectrochemical device for generating at least one of the carbon dioxide reduction products selected from the group consisting of methane, carbon monoixide, and formic acid from the carbon dioxide dissolved in the liquid, wherein
the photoelectrochemical device comprises:
a cathode chamber for storing a first electrolyte solution containing the carbon dioxide dissolved in the liquid;
an anode chamber for storing a second electrolyte solution;
a solid electrolyte membrane interposed between the cathode chamber and the anode chamber;
a cathode electrode provided in the cathode chamber so as to be in contact with the first electrolyte solution;
an anode electrode provided in the anode chamber so as to be in contact with the second electrolyte solution; and
an external power supply for applying a negative voltage and a positive voltage to the cathode electrode and the anode electrode, respectively;
(b) putting an organic waste and the methane bacteria into the fermenter;
(c) decomposing the organic waste by the action of the methane bacteria to generate the biogas containing the carbon dioxide and the methane;
(d) dissolving the carbon dioxide contained in the generated biogas in a liquid; and
(e) reducing the carbon dioxide on the cathode electrode by applying a voltage from the external power supply through the anode electrode and the cathode electrode to the liquid in which the carbon dioxide has been dissolved, so as to give at least one of the carbon dioxide reduction products selected from the group consisting of methane, carbon monoxide, and formic acid on a surface of the cathode electrode.

7. The method according to claim 6, wherein
the cathode electrode is formed of copper or a compound thereof; and
the carbon dioxide reduction product is methane.

8. The method according to claim 6, wherein
the cathode electrode is formed of gold, silver, or an alloy thereof; and
the carbon dioxide reduction product is carbon monoxide.

9. The method according to claim 6, wherein
the cathode electrode is formed of indium, tin, or an alloy thereof; and
the carbon dioxide reduction product is formic acid.
